(19) United States
(12) Patent Application Publication
(10) Pub. No.: US 2006/0185678 A1
(43) Pub. Date: Aug. 24, 2006

(54) Devices for delivering agents to a vaginal tract

(76) Inventors: Lance J. Bronnenkant, Snyder, NY (US); Anu Mahashabde, Kendall Park, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/346,560

(22) Filed: Feb. 3, 2006

Related U.S. Application Data
(60) Provisional application No. 60/649,078, filed on Feb. 3, 2005.

(51) Int. Cl. A61F 6/02

(52) U.S. Cl. 128/842

(57) ABSTRACT

The present invention is directed to devices for delivering agents to a vaginal tract, a device of the invention comprises a non-porous polymeric material substantially impermeable to an environment of the vaginal tract, the non-porous polymeric material forming a closed envelope which forms an internal space, wherein the closed envelope contains two or more distinct apertures.

### CROSS REFERENCE TO RELATED APPLICATIONS

This application claims the benefit of the filing date of U.S. Application No. 60/649,078, filed Feb. 3, 2005, the contents of which are hereby incorporated by reference in its entirety.

### BACKGROUND OF THE INVENTION

1. Field of the Invention

The present invention relates to devices that can deliver agents to a vaginal tract. Especially, the present invention relates to a device for delivering an agent to a vaginal tract, the device comprising a non-porous polymeric material substantially impermeable to an environment of the vaginal tract, the non-porous polymeric material forming a closed envelope which forms an internal space, wherein the closed envelope contains two or more distinct apertures.

2. Background Art

Devices for delivering an agent to a vaginal tract have been previously described. For example, U.S. Pat. No. 196,979 describes a vaginal medicated-ring device. U.S. Pat. No. 3,545,439 describes an intravaginal ring-shaped device made of a solid polymer, the device containing a drug that is released by diffusion to the vagina. U.S. Pat. No. 3,920,805 describes a polymeric device with a solid, non-medicated central core and a medicated coating on the core which releases drug by diffusion. U.S. Pat. No. 4,012,496 describes a medicament-free vaginal ring having an encircling indentation with another smaller, medicament-containing ring placed in the indentation. Other vaginal medicament dispensing means are disclosed in U.S. Pat. Nos. 3,991,760; 3,924,622; 3,995,633; 4,155,991; 4,292,964; 4,286,587; 4,601,893; 4,830,860; and 5,788,977.

The vaginal devices described above are useful for their intended purposes and represent valuable contributions to the vaginal dispensing art. However, a need exists for delivery of an agent to the vaginal tract wherein the rate of delivery of the agent is not solely dependent on the diffusion of the agent from the vaginal device.

### BRIEF SUMMARY OF THE INVENTION

The present invention is directed to a vaginal device comprising a non-porous polymeric material substantially impermeable to an environment of the vaginal tract, the non-porous polymeric material forming a closed envelope which forms an internal space, wherein the closed envelope contains two or more distinct apertures.

The present invention is also directed to a method of administering an agent to a subject in need of the agent, the method comprising administering the device of the present invention to the subject.

The present invention is also directed to a method of making a device for delivering an agent to a vaginal tract, the method comprising forming a closed envelope comprising a non-porous polymeric material substantially impermeable to an environment of the vaginal tract, wherein the closed envelope forms an internal space; and forming two or more distinct apertures in the closed envelope.

The present invention is also directed to a method of making a device for delivering an agent to a vaginal tract, the method comprising: (a) forming a closed envelope comprising a non-porous polymeric material substantially impermeable to an environment of the vaginal tract, wherein the closed envelope forms an internal space; (b) forming two or more distinct apertures in the closed envelope of (a); and (c) placing a matrix containing an agent into the internal space of (a), thus forming a matrix encased by the closed envelope.

### BRIEF DESCRIPTION OF THE DRAWINGS

FIG. 1A is a schematic representation of the top view cross-section of a vaginal ring according to the invention. FIG. 1A consists of seven apertures [1] extending through the non-porous polymeric material [2] which forms a closed envelope which forms an internal space [3]. FIG. 1B represents the top view cross-section of a vaginal ring containing three separate internal spaces.

FIG. 2 is a schematic representation of various 3-dimensional shapes of apertures according to the invention (FIGS. 2A-2D). In FIG. 2A, [4] represents the surface area of the aperture that faces the vaginal environment;

[5] represents the surface area of the aperture that faces the internal space of the device; and [6] represents a cut-out section of the non-porous polymeric material which forms a closed envelope.

FIG. 3 represents the effect of various aperture sizes on release rates of a drug as described in Example 5. The release rates were determined using vaginal rings with (a) about 380 μm diameter apertures (filled circles), (b) about 250 μm diameter apertures (circles), (c) about 180 μm diameter apertures (filled triangles), or (d) about 100 μm diameter apertures (triangles).

FIG. 4 represents the effect of various drug and matrix concentrations on release rates from a vaginal ring containing 100 μm apertures. The drug and matrix concentrations included (a) 12% danazol, 8.8% hydroxyethylcellulose (HEC), and 79.2% glycerin (filled circles), (b) 6% danazol, 10.4% HEC, and 83.6% glycerin (circles), and (c) 3% danazol, 12% HEC, and 85% glycerin (filled triangles).

### DETAILED DESCRIPTION OF THE INVENTION

The present invention is directed to a vaginal device comprising a non-porous polymeric material substantially impermeable to an environment of the vaginal tract, the non-porous polymeric material forming a closed envelope which forms an internal space, wherein the closed envelope contains two or more distinct apertures.

The present invention is also directed to a method of making a device for delivering an agent to a vaginal tract, the method comprising (a) forming a closed envelope comprising a non-porous polymeric material substantially impermeable to an environment of the vaginal tract, wherein the closed envelope forms an internal space, and (b) forming two or more distinct apertures in the closed envelope of (a).

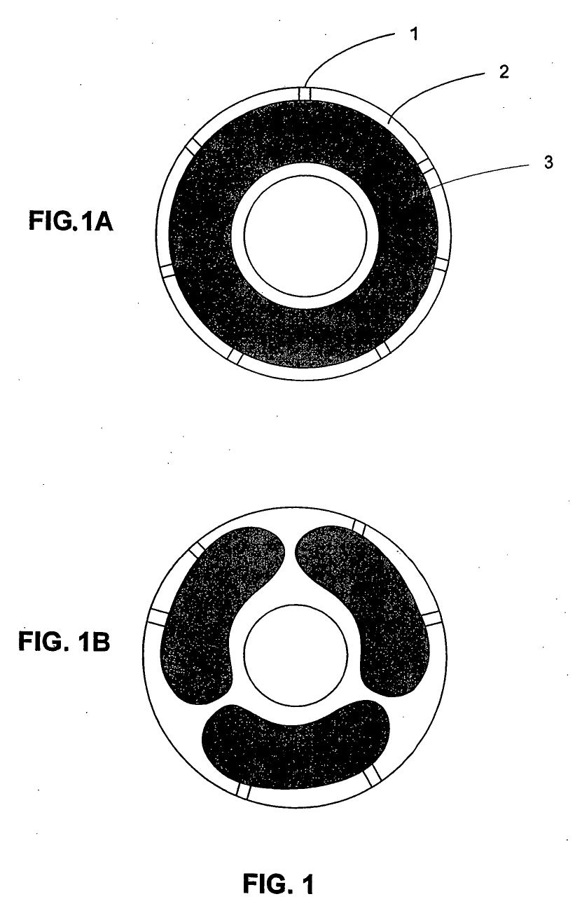

In some embodiments, the closed envelope can form a single internal space. In some embodiments, the closed envelope can form multiple internal spaces (see Fig. 1B), i.e., the closed envelope can form greater than one internal space. In some embodiments, the closed envelope can form one to six internal spaces. In some embodiments, the closed envelope can form two to four internal spaces.

In some embodiments wherein the closed envelope forms multiple internal spaces, each internal space can contain one or more apertures. In some embodiments wherein the closed envelope forms multiple internal spaces, each internal space can contain the same number of apertures. In some embodiments wherein the closed envelope forms multiple internal spaces, the number of apertures in each internal space can vary. For example, for a closed envelope that forms three internal spaces, one internal space can have 5 apertures, one internal space can have 8 apertures, and one internal space can have 12 apertures.

In some embodiments wherein the closed envelope forms multiple internal spaces, the apertures in a particular internal space can be a different size than the apertures of another internal space formed from the same closed envelope. For example, for a closed envelope that forms three internal spaces, one internal space can have apertures with an average diameter of about 200 μm, one internal space can have apertures with an average diameter of about 300 μm, and one internal space can have apertures with an average diameter of about 500 μm.

In some embodiments, a matrix is contained within the internal space.

The term “matrix” refers to any carrier substance capable of suspending or solubilizing an agent. The matrix can be in various physical states. For example, the matrix can be, but is not limited to a solid, semi-solid, or viscous liquid. Various matrices can be used. In some embodiments, the matrix can be, but is not limited to, a lipophilic or hydrophilic polymer matrix, or combinations thereof. In some embodiments, the matrix can be a hydrogel, viscous oil, glass, or powder. In some embodiments, the matrix can be amorphous or crystalline. In some embodiments, the matrix is bioerodible. In some embodiments, the matrix further comprises an agent capable of diffusing from the matrix into the vaginal tract. In some embodiments, a single internal space can comprise two or more different matrices. The term “agent” refers to a drug, protein, hormone, vitamin, nutritional supplement, or any other substance intended for use in the treatment, mitigation, cure or prevention of a disease or any other medical condition. In some embodiments, the matrix is an ion-exchange resin to which the agent is bound. In some embodiments, the agent is covalently linked to the matrix. In some embodiments, an agent covalently linked to the matrix is released via an interaction with vaginal fluid. In some embodiments in a device with multiple internal spaces, each internal space contains the same matrix. In some embodiments in a device with multiple internal spaces, the internal spaces can contain the same or different matrices.

The agents can be released at various rates. The release rate can be dependent on various factors. In some embodiments, the release rate is dependent on the matrix formulation, the number of apertures, the size of the apertures, the shape of the apertures, the composition of the non-porous polymeric material, the thickness of the non-porous polymeric material, the environment of use, and/or the agent being released. In some embodiments, the agent is released from the device to an environment of the vaginal tract at a rate of about 0.001 mg/day to about 100 mg/day. In some embodiments, the agent is released from the device to the environment of the vaginal tract at a rate of about 0.01 mg/day to about 10 mg/day. In some embodiments, the agent is released from the device at a substantially higher rate once the device is placed in the vaginal tract. In some embodiments, the agent is not substantially released from the device of the present invention prior to placement in the vaginal tract.

In some embodiments, the agent can be dissolved in the matrix to form a solution. Alternatively, the agent can be present heterogeneously, e.g., in particle form, in the matrix, thus forming a suspension. In some embodiments, the agent has a particle size of about 0.01 μm to about 1000 μm. In some embodiments, the agent has a particle size of about 0.1 μm to about 10 μm.

In the present invention, a non-porous polymeric material forms a closed envelope which forms an internal space. A closed envelope is one in which the envelope completely wraps around the internal space. In some embodiments, the closed envelope has a relatively constant thickness. In some embodiments, the closed envelope has a thickness of about 5 μm to about 1 cm, about 10 μm to about 0.5 cm, or about 50 μm to about 200 μm.

The term “non-porous” refers to the absence of pores of sufficient size to essentially prohibit passage of the vaginal fluid through the polymeric material to the matrix. Thus, only vaginal fluid that passes through the apertures can enter the internal space of the present invention. In some embodiments, the non-porous polymeric material is permeable or semi-permeable to the agent. In some embodiments, the non-porous polymeric material is not permeable or semi-permeable to the agent. In some embodiments, the non-porous polymeric material can be bioerodible.

Various non-porous materials can be used. In some embodiments, the non-porous polymeric material is a thermoplastic material, thermoplastic elastomeric material, thermosetting material or combinations thereof. In some embodiments, the non-porous material is not an elastomeric material, e.g., silicone.

In some embodiments, the non-porous polymeric material is a thermoplastic material. The thermoplastic material is a plastic material capable of softening when heated and hardening again when cooled. Suitable thermoplastic materials include, but are not limited to, a thermoplastic olefin blend, a crosslinked polymer, a copolymer, a block copolymer, and combinations thereof. In some embodiments, the thermoplastic polymer can be a block copolymer comprised of polypropylene and ethylene propylenediene rubber, e.g., Santoprene® thermoplastic.

In some embodiments, the non-porous polymeric material is a thermoplastic elastomeric material. Thermoplastic elastomeric materials of the present invention are any materials that possess excellent elasticity and resilience at ambient temperatures, without the need for vulcanization to develop rubberlike elasticity. Suitable thermoplastic elastomeric materials include, but are not limited to, polyurethanes, copolyesters and blends of ethylene-propylene copolymers with polypropylene. In some embodiments, the thermoplastic elastomeric material can be, but is not limited to, a thermoplastic olefin blend, an ionomer, a block copolymer, or combinations thereof. In some embodiments, the thermoplastic elastomeric material is a styrene-ethylene-butylene modified block copolymer, e.g., C-Flex® tubing (Cole-Parmer, Vernon Hills, Ill.).

Various block copolymers can be used. In some embodiments, the block copolymer is a styrene-butadiene block copolymer. In some embodiments, the block copolymer comprises polypropylene and ethylene-propylene diene rubber. In some embodiments, the block copolymers comprise styrene with butadiene or isoprene.

In some embodiments, the non-porous polymeric material is a thermosetting material. A thermosetting material is any material that changes irreversibly under the influence of heat from a fusible and soluble material into one which is infusible and insoluble through the formation of a covalently linked, thermally stable network. Suitable thermosetting materials include, but are not limited to, crosslinked polymers, copolymers, block copolymers, and combinations thereof.

The closed envelope of the present invention can be in any shape, as long as it is suitable for placement in the vaginal tract. In some embodiments, the closed envelope is a tampon. In some embodiments, the closed envelope is annular in shape. For example, in some embodiments, the closed envelope is an intravaginal ring.

The device of the present invention contains distinct apertures. The term “distinct” indicates that the perimeters of the apertures are not in physical contact with each other. The term “aperture” refers to holes or openings in the envelope that extend through the thickness of the envelope to connect the internal space to the environment outside the envelope. The distinct apertures of the present invention can be produced by methods known to those in the art. For example, the distinct apertures can be formed by laser drilling, mechanical pressing, or mechanical drilling. Various types of laser drill apparatus can be used. For example, a LPM model laser drill from LasX (Minneapolis, Minn.) or a laser drill manufactured by PRECO Laser Systems (Somerset, Wis.).

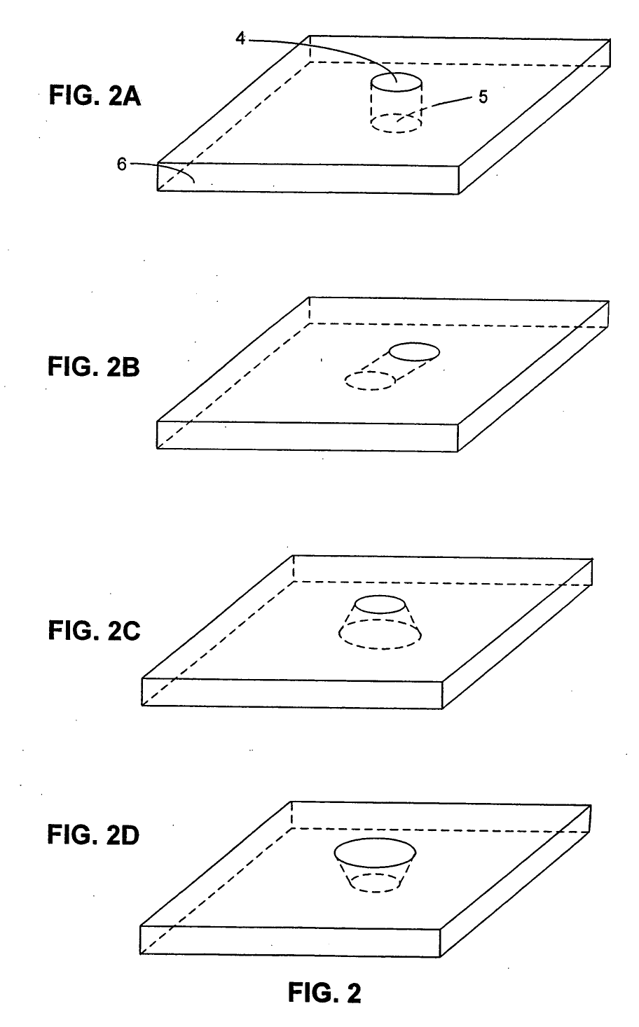

The apertures can be of various shapes. In some embodiments, the shape of the perimeter of the face of the aperture facing the environment of the vaginal tract can be, but is not limited to, an amorphous shape, substantially circular, substantially oval, substantially in the form of a polygon or combinations thereof. A polygon includes, but is not limited to, a shape consisting of between three to about 10 angles, e.g., a substantially triangular polygon, substantially rectangular polygon, substantially pentangular polygon, etc. FIG. 2 displays examples of schematic representations of three-dimensional shapes of apertures that are possible in some embodiments of the present invention. In some embodiments, at least one of the apertures that extends through the envelope is substantially perpendicular to the outside surface of the envelope, and thus the surface area facing the vaginal tract is approximately the same size as, and is aligned with, the surface area facing the internal space (FIG. 2A). In some embodiments, at least one of the apertures that extends through the envelope is not substantially perpendicular to the outside surface of the envelope but the surface area facing the vaginal tract is approximately the same size as the surface area facing the internal space (FIG. 2B). In some embodiments, at least one of the distinct apertures has a larger surface area facing the internal space than a surface area facing the vaginal tract, thus forming an essentially semi-conical shape (FIG. 2C). In some embodiments, at least one of the apertures has a larger surface area facing the vaginal tract than the surface area facing the internal space, thus forming an essentially inverted semi-conical shape (FIG. 2D).

Various numbers of apertures can be present in the closed envelope of the present invention. In some embodiments, the closed envelope can comprise two or more, or three or more, distinct apertures. In some embodiments, the closed envelope can comprise four or more distinct apertures. In some embodiments, the closed envelope can comprise three to about 1000 distinct apertures. In some embodiments of the present invention, the closed envelope comprises greater than 10 distinct apertures. In some embodiments, the closed envelope has about 30 distinct apertures to about 60 distinct apertures. In some embodiments for devices with multiple internal spaces, the number of apertures connecting an internal space with the outside environment can vary for each internal space. In some embodiments, the variation in the number of apertures connected to different internal spaces provides for different release rates of active agent from the internal spaces.

In the present invention, at least one of the apertures has a length and a width having a ratio of less than about 20:1. The term “length” refers to the longest straight distance across the face of the aperture facing the vaginal tract.

The term “width” refers to the distance across the face of the aperture facing the vaginal tract, wherein the distance measured is the largest straight distance perpendicular to the length. In some embodiments, the length and width of at least one of the distinct apertures has a ratio of less than about 10:1. In some embodiments, the length and width of at least one of the distinct apertures has a ratio of less than about 5:1.

Various sizes of apertures can be used in the present invention. In some embodiments, at least one distinct aperture has a diameter of about 1 μm to about 1 mm. In some embodiments, at least one distinct aperture has a diameter of about 10 μm to about 1000 μm. In some embodiments, at least one distinct aperture has a diameter of about 100 μm to about 500 μm. In some embodiments, at least one distinct aperture has a diameter of about 300 μm to about 400 μm. In some embodiments, the average diameter of the distinct apertures is about 1 μm to about 1000 μm. In some embodiments, the average diameter of the distinct aperture is about 100 μm to about 500 μm. In some embodiments, the average diameter of the distinct aperture is about 300 μm to about 400 μm. The term “diameter” refers to the length of the longest straight line that contacts two sides of the aperture that passes through the geometric center of the surface area of the aperture facing the vaginal tract.

The apertures of the present invention can encompass various amounts of the surface area of the device. In some embodiments, the distinct apertures have a total surface area that covers about 0.01% to about 50% of the device. In some embodiments, the distinct apertures have a total surface area that covers about 0.05% to about 10% of the device. In some embodiments, the distinct apertures have a total surface area that covers about 0.1% to about 1% of the device. The term “surface area” refers to the area of the face of the aperture facing the vaginal tract. The term “total surface area” refers to the summation of the surface area facing the vaginal tract of all the apertures on a device divided by the total surface area of the device. For example, if a device has a total surface area equal to 100 mm , and that device has 4 apertures each with a surface area of 1 mm2 (4 mm2 total surface area), then the apertures would have a total surface area that covers 4% of the device.

The apertures can be located at various locations on the device of the present invention. In some embodiments, the apertures are evenly dispersed over the surface of the device. In some embodiments, the apertures are grouped, or clustered, in one or more locale on the surface of the device. In some embodiments, the apertures are randomly dispersed over the surface of the device. In some embodiments, the apertures are arranged in a pattern on the device, e.g., in a vaginal ring, the apertures can be arranged in one or more lines that extend around the perimeter of the vaginal ring.

In the present invention, a second agent different from the agent in the matrix can be present in the device. In some embodiments, the second agent is contained in one or more apertures. In some embodiments, the second agent is contained in the non-porous polymeric material. In some embodiments, the second agent is in the matrix. In some embodiments, the second agent prevents clogging of the distinct apertures. In some embodiments, the second agent is a chelating agent. Various chelating agents are known in the art. A chelating agent is a substance whose molecules can form several bonds to a metal ion. In other words, a chelating agent is a multidentate ligand. Suitable chelating agents include, but are not limited to, citric acid, amidomalonic acid (Ama), gluconic acid, ethylenediaminetetraacetic acid (EDTA, edetic acid), associated salts thereof, and combinations thereof. In some embodiments, the chelating agent is EDTA.

In some embodiments, the second agent is an antimicrobial agent. An antimicrobial agent is a substance that destroys or inhibits the growth of microorganisms. Antimicrobial agents include, but are not limited to, antibacterial drugs, antiviral agents, antifungal agents, and antiparisitic drugs. Suitable antimicrobial agents include, but are not limited to, triclosan, chlorhexidine, silver sulfadiazine, silver ions, benzalkonium chloride, zinc pyrithione, broad-spectrum antibiotics, antiseptic agents and iodine.

In some embodiments, three to ten different agents can be present in the device of the present invention. Each of these agents can be present, independent of each other, in the matrix, the internal space, the aperture and/or the non-porous polymeric material.

As described herein, the descriptive terms “facing the vaginal tract” and “faces the vaginal tract” refer to the outside of the device that is exposed to the environment of the vaginal tract when the device in placed in a vaginal tract. These descriptive terms are solely used to describe a location or feature of the present invention relative to other locations or features on the present inventions and are not meant to limit the invention when the invention is not in a vaginal tract. Thus, e.g., “the surface area of an aperture facing the vaginal tract” refers to the surface area of the aperture when the device of the present invention is in the vaginal tract, and also to the same surface area when the device of the present invention is not in a vaginal tract.

As used herein, “about” refers to plus or minus 10% of the indicated number or numbers. For example, “about 10 μm” indicates a range of 9 μm to 11 μm; and “about 10:1” indicates a range of 9:1.1 to 11:0.9.

The present invention is also directed to a method of administering an agent to a subject in need of the agent, the method comprising administering the device containing the agent as described herein to the subject. The term “subject” refers to any female. In some embodiments, the subject is a mammal. In some embodiments, the subject is a human.

The device of the present invention can be delivered to a subject in need thereof for various periods of time. In some embodiments, the administering of the device occurs continuously for about one month to about one year. The term “continuously” refers to administration of the device for the indicated time, inasmuch as the therapeutic or beneficial effect of the agent continues to be manifest in the subject being treated without interruption. Thus, it is within the scope of the invention for the device to be removed from the vaginal tract for a period of time as long as the beneficial effects of the agent continue while the device is not present in the vaginal tract. In some embodiments, the administering of an agent occurs continuously for, but not limited to, about one hour to about one year, about one day to about 90 days, or about 7 days to about 30 days.

The present invention is also directed to a method of making a device for delivering an agent to a vaginal tract, the method comprising (a) forming a closed envelope comprising a non-porous polymeric material substantially impermeable to an environment of the vaginal tract, wherein the closed envelope forms an internal space, and (b) forming two or more distinct apertures in the closed envelope of (a). In some embodiments, the method further comprises (c) placing a matrix containing an agent into the internal space of (a), thus forming a matrix encased by the closed envelope.

In some embodiments, the invention is directed to a method of making a device for delivering an agent to a vaginal tract, the method comprising (a) forming a closed envelope comprising a non-porous polymeric material substantially impermeable to an environment of the vaginal tract, wherein the closed envelope forms an internal space, (b) forming two or more distinct apertures in the closed envelope of (a), and (c) placing a matrix containing an agent into the internal space of (a), thus forming a matrix encased by the closed envelope.

### EXAMPLE 1

Two different fatty acid matrices were prepared: (1) a 10% formulation which contained 10% 17α-ethynyl-17β-hydroxy-4-androsteno[2,3-d]isoxazole and 90% Gelucire® 50/13 (Formulation I), and (2) a 30% formulation which contained 30% 17α-ethynyl-17β-hydroxy-4-androsteno[2,3-d]isoxazole and 70% Gelucire® 50/13 (Formulation II).

### EXAMPLE 2

Two different hydrogel matrices were prepared. A 10% Formulation (Formulation III) and a 30% Formulation (Formulation IV) are described in Table 1:

TABLE 1

| | | |
|---|---|---|
| | 10% Formulation | 30% Formulation |
| Ingredients | (% w/w) | (% w/w) |

Phase A

| | | |
|---|---|---|
| Water, purified | 24.50 | 16.00 |
| Carbomer 971 P | 0.50 | 0.50 |

Phase B

| | | |
|---|---|---|
| Polyethylene glycol 400 | 20.00 | 15.00 |
| Propylene glycol | 20.00 | 15.00 |
| Natrosol 250 HHX | 2.50 | 2.50 |
| (hydroxyethylcellulose) | | |

Phase C

| | | |
|---|---|---|
| 17α-ethynyl-17β-hydroxy-4- | 10.00 | 30.00 |
| androsteno[2,3-d]isoxazole | | |
| ethanol | 12.00 | 15.00 |
| Ethanol rinse | 5.00 | 3.00 |
| Water rinse | 5.00 | 3.00 |

### EXAMPLE 3

Various numbers of holes of varying sizes were placed in a section of C-Flex tubing by using a LPM model laser drill from LasX (Minneapolis, Minn.). A matrix from Example 1 or Example 2 was then placed in a tube section. The ends of the tube section were then capped to prevent the matrix from being released from the end of the tube section. Table 2 summarizes the size and number of holes per tube section.

TABLE 2

| Tube | Matrix | Hole | # of |
|---|---|---|---|
| Section | Formulation | size (μm) | holes |
| A | Formulation I | 130 | 30 |
| B | Formulation I | 410 | 30 |
| C | Formulation I | 410 | 60 |
| D | Formulation II | 130 | 30 |
| E | Formulation II | 410 | 30 |
| F | Formulation II | 410 | 60 |
| G | Formulation III | 130 | 30 |
| H | Formulation III | 410 | 30 |
| I | Formulation III | 410 | 60 |
| J | Formulation IV | 130 | 30 |
| K | Formulation IV | 410 | 30 |
| L | Formulation IV | 410 | 60 |

### EXAMPLE 4

The prepared tube sections A-L from Example 3 were placed in a shaker bath containing 500 mL of 0.05 M sodium dodecyl sulfate (SDS) dissolution media at 37° C. The bath shaker speed was 99 strokes per minute. A 10 mL sample of dissolution media was taken at days 1, 2, 3, 4, 7, 11, 14, 21, and 28. Upon taking a sample, 10 mL of fresh 0.05 M SDS was added to the dissolution media. The amount of drug released (mg/day) was calculated for each tube section for each of the indicated days. Results (drug released (mg/day)) for tube sections containing fatty acid matrices (Formulations I and II) are found in Table 3. Results (drug released (mg/day)) for tube sections containing hydrogel matrices (Formulations III and IV) are found in Table 4.

TABLE 3

Tube Section

| Day | A | B | C | D | E | F |
|---|---|---|---|---|---|---|
| 1 | 1.24 | 2.17 | 4.52 | 0.51 | 2.36 | 3.43 |
| 2 | 1.69 | 2.05 | 6.17 | 0.63 | 5.36 | 12.47 |
| 3 | 2.50 | 6.92 | 12.11 | 2.69 | 8.19 | 17.75 |
| 4 | 4.18 | 9.54 | 7.25 | 8.07 | 17.80 | 13.75 |
| 7 | 2.62 | 4.60 | 4.28 | 6.72 | 8.99 | 11.49 |
| 11 | 4.40 | 4.90 | 2.50 | 8.61 | 9.66 | 10.74 |
| 14 | 4.05 | 1.36 | 1.35 | 11.48 | 7.37 | 5.37 |
| 21 | 1.21 | 0.49 | 0.76 | 2.81 | 2.28 | 2.03 |

TABLE 4

Tube Section

| Day | G | H | I | J | K | L |
|---|---|---|---|---|---|---|
| 1 | 0.35 | 0.59 | 1.28 | 0.64 | 0.68 | 1.49 |
| 2 | 0.04 | 2.69 | 6.90 | 0.2 | 0.09 | 2.01 |
| 3 | 0.57 | 4.95 | 8.19 | 0.4 | 1.11 | 7.46 |
| 4 | 0.59 | 5.61 | 6.42 | 0.5 | 2.82 | 10.19 |
| 7 | 1.38 | 3.67 | 2.20 | 0.71 | 4.67 | 6.67 |
| 11 | 1.97 | 1.01 | 0.05 | 0.98 | 4.79 | 1.31 |
| 14 | 2.60 | 0.42 | 0.20 | 1.10 | 2.23 | 0.71 |
| 21 | 3.22 | 0.07 | 0.17 | 0.31 | 0.11 | 1.01 |

### EXAMPLE 5

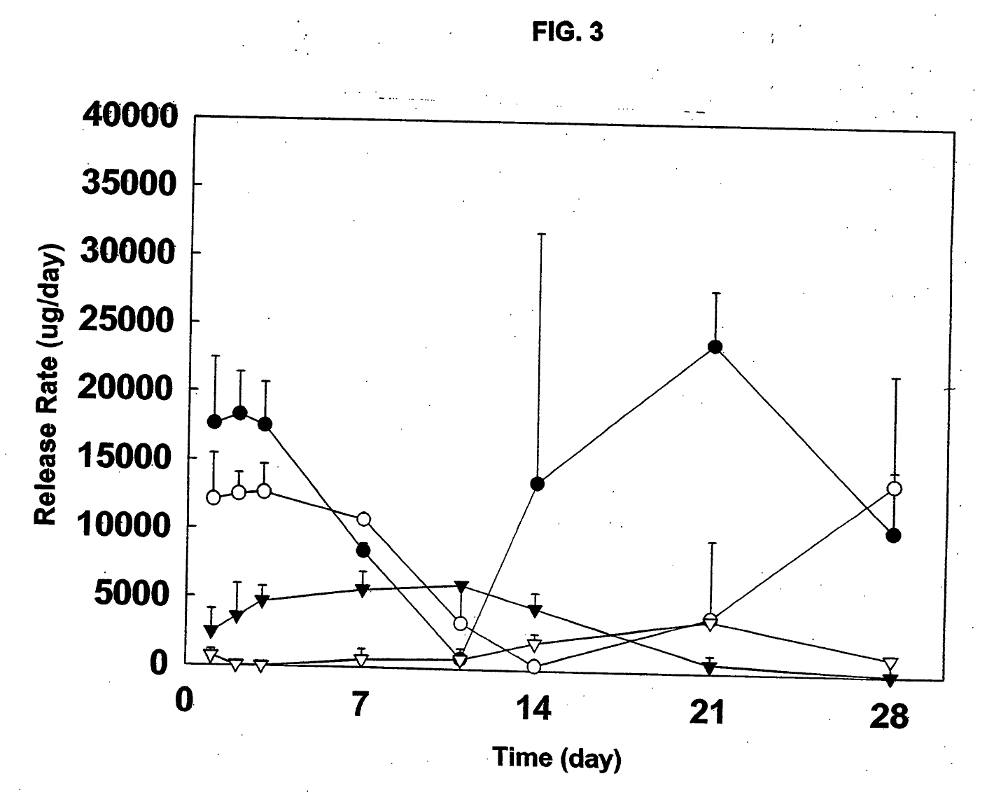

The effect of various aperture sizes on release rates was determined. An 8 mm vaginal ring containing an internal space was fabricated by a Kuntz injection molding instrument at 100° C. (Kuntz Mfg. Co., Inc., Santa Ana, Calif.). The temperature was then changed to 120° C., and the vaginal ring was molded at 120° C. for 8 minutes and then cooled for 6 minutes. Thirty apertures were placed in the vaginal ring by the use of a laser drill. The size of the apertures in each of four vaginal rings was either 380 μm, 250 μm, 180 μm or 100 μm. A non-aqueous gel matrix containing danazol was prepared by mixing 8.8% Natrosol 250 HHX (hydroxyethylcellulose; HEC), 79.2% glycerin and 12% danazol (w/w) for 5 minutes. The matrix was then placed into a 16 gauge syringe and injected into the internal space of the vaginal ring through one of the laser drilled holes. The prepared vaginal rings were then placed in a shaker bath containing 500 mL of 0.05 M sodium dodecyl sulfate (SDS) dissolution media at 37° C. The bath shaker speed was 99 strokes per minute. A 10 mL sample of dissolution media was taken at day 1, 2, 3, 7, 11, 14, 21, and 28. Upon taking a sample, 10 mL of fresh 0.05 M SDS was added to the dissolution media. The amount of drug released (μg/day) was calculated for each vaginal ring for each of the indicated days. Results (drug released (μg/day)) for the vaginal rings containing the non-aqueous gel are found in FIG. 3. The values in FIG. 3 represent an average of experiments done in triplicate and represent the release rate in μg/day.

### EXAMPLE 6

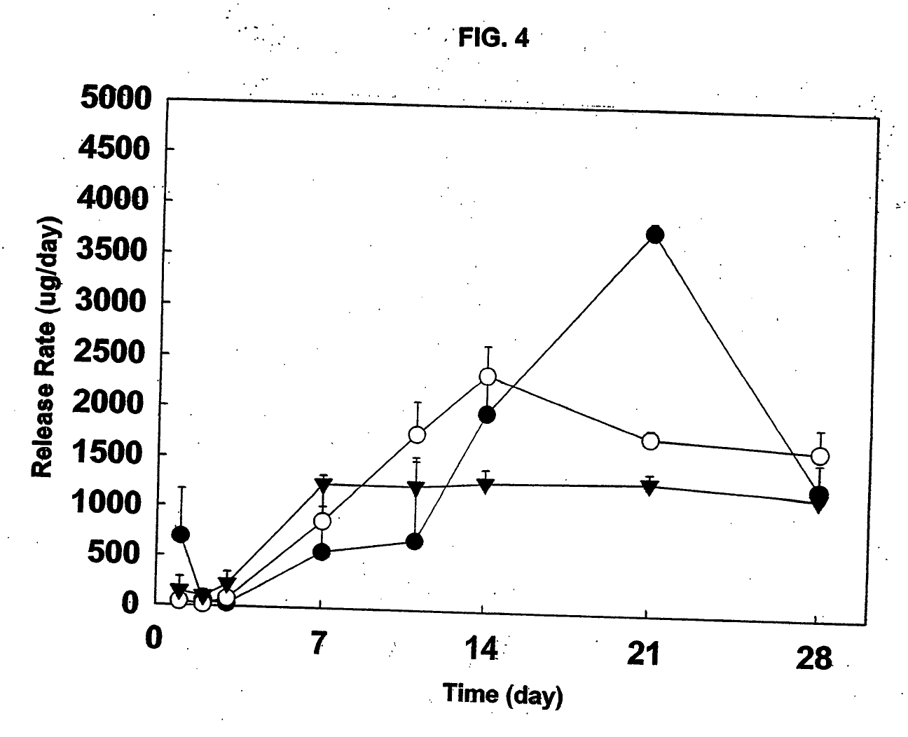

The effect of various concentrations of a drug on the release rate of that drug was determined for a vaginal ring containing 30 apertures, each aperture with a diameter of about 100 μm. Three different matrices were investigated. Matrix 1 contained 12% danazol, 8.8% HEC, and 79.2% glycerin. Matrix 2 contained 6% danazol, 10.4% HEC and 83.6% glycerin. Matrix 3 contained 3% danazol, 12% HEC and 85% glycerin. Each vaginal ring was then placed in a shaker bath containing 500 mL of 0.05 M sodium dodecyl sulfate (SDS) dissolution media at 37° C. The bath shaker speed was 99 strokes per minute. A 10 mL sample of dissolution media was taken at day 1, 2, 3, 7, 11, 14, 21, and 28. Upon taking a sample, 10 mL of fresh 0.05 M SDS was added to the dissolution media. The amount of drug released (μg/day) was calculated for each vaginal ring for each of the indicated days. Results (drug released (μg/day)) for the vaginal rings containing the various concentrations of danazol are found in FIG. 4. The values in FIG. 4 are an average of experiments done in triplicate and represent the release rate in μg/day.

These examples illustrate some possible embodiments of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. A vaginal device comprising a non-porous polymeric material substantially impermeable to an environment of the vaginal tract, the non-porous polymeric material forming a closed envelope which forms an internal space, wherein the closed envelope contains two or more distinct apertures.

2. The device of claim 1, wherein the closed envelope forms greater than one internal space.

3. The device of claim 1, wherein the closed envelope forms from one to six internal spaces.

4. (canceled)

5. The device of claim 1, further comprising a matrix contained within the internal space.

6. (canceled)

7. The device of claim 5, wherein the matrix further comprises an agent capable of diffusing from the matrix into the vaginal tract.

8. (canceled)

9. (canceled)

10. (canceled)

11. The device of claim 7, wherein the agent is released from the device to the environment of the vaginal tract at a rate of about 0.001 mg/day to about 100 mg/day.

12. (canceled)

13. (canceled)

14. The device of claim 7, wherein the distinct apertures contain a second agent different from the agent in the matrix.

15. The device of claim 14, wherein the second agent prevents clogging of the distinct apertures.

16. (canceled)

17. (canceled)

18. (canceled)

19. The device of claim 7, wherein the non-porous polymeric material comprises a second agent different from the agent in the matrix.

20. The device of claim 19, wherein the second agent is an agent that prevents clogging of the distinct apertures.

21. (canceled)

22. (canceled)

23. (canceled)

24. A method of administering an agent to a subject in need of the agent, the method comprising administering the device of claim 7 to the subject.

25. The method of claim 24, wherein the administering of an agent occurs continuously for about one hour to about one year.

26. (canceled)

27. (canceled)

28. The device of claim 1, wherein at least one of the distinct apertures has a length and a width having a ratio of less than 20:1.

29. The device of claim 1, wherein the closed envelope has a thickness of between about 5 μm to about 1 cm.

30. (canceled)

31. (canceled)

32. The device of claim 1, wherein the non-porous polymeric material is a thermoplastic material, thermoplastic elastomeric material, thermosetting material or combinations thereof.

33. (canceled)

34. The device of claim 32, wherein the thermoplastic material is a thermoplastic olefin blend, a crosslinked polymer, a copolymer, a block copolymer, or combinations thereof.

35. (canceled)

36. (canceled)

37. (canceled)

38. (canceled)

39. (canceled)

40. (canceled)

41. (canceled)

42. (canceled)

43. The device of claim 1, wherein the closed envelope is annular in shape.

44. The device of claim 43, wherein the closed envelope is an intravaginal ring.

45. The device of claim 1, wherein the distinct apertures are formed by laser drilling.

46. (canceled)

47. (canceled)

48. (canceled)

49. (canceled)

50. (canceled)

51. (canceled)

52. (canceled)

53. (canceled)

54. A method of making a device for delivering an agent to a vaginal tract, the method comprising:
(a) forming a closed envelope comprising a non-porous polymeric material substantially impermeable to an environment of the vaginal tract, wherein the closed envelope forms an internal space; and
(b) forming two or more distinct apertures in the closed envelope of (a).

55. The method of claim 54 further comprising:
(c) placing a matrix containing an agent into the internal space of (a), thus forming a matrix encased by the closed envelope.

56. The method of claim 55 wherein the distinct apertures are formed by laser drilling.

57. A method of making a device for delivering an agent to a vaginal tract, the method comprising:
(a) forming a closed envelope comprising a non-porous polymeric material substantially impermeable to an environment of the vaginal tract, wherein the closed envelope forms an internal space;
(b) forming two or more distinct apertures in the closed envelope of (a); and
(c) placing a matrix containing an agent into the internal space of (a), thus forming a matrix encased by the closed envelope.
